(19) United States
(12) Patent Application Publication
(10) Pub. No.: US 2009/0078320 A1
(43) Pub. Date: Mar. 26, 2009

(54) Steam exhaust assembly

(76) Inventor: Paul Marttila, Milton (CA)

Correspondence Address: CLARK & BRODY, 1090 VERMONT AVENUE, NW, SUITE 250, WASHINGTON, DC, 20005, US

(21) Appl. No.: 11/902,813

(22) Filed: Sep. 26, 2007

(51) Int. Cl. F17D 1/12; A61L 2/07; F16T 1/36

(52) U.S. Cl. 137/2; 137/177; 422/26

(57) ABSTRACT

There is provided an apparatus for condensing and removing exhaust steam from sterilization equipment. The apparatus comprises a primary exhaust vessel and condensing means which accepts and condenses exhaust steam from sterilization equipment. A secondary exhaust vessel, connected to the primary exhaust vessel, provides for further condensing and elimination of exhaust steam. The apparatus may include a drain system for disposing of condensate from the secondary exhaust vessel.

### FIELD OF THE INVENTION

The present invention relates to a steam exhaust assembly for use with sterilization equipment which employs steam for sterilizing and superheated steam for drying of medical or dental instruments.

### BACKGROUND OF THE INVENTION

Sterilization processes which are used to sterilize medical and dental instruments and the like typically use steam in the sterilization of such instruments. Some sterilization processes further use superheated steam in the drying of such instruments. Sterilization equipment, such as cassette autoclaves, also are being made more compact in order to fit into smaller environments, such as those found in a dental office or medical clinic setting. Since these devices may emit significant amounts of heat and steam during the sterilization process, there is a need to remove the excess heat and steam in a controlled, effective and efficient manner rather than releasing the heat and steam into the ambient environment. Further, building codes also may regulate the discharge and disposal of exhaust steam thereby requiring a removal of excess heat and steam in a controlled manner.

Currently in autoclaves that do not recycle exhaust water, a condensate bottle or receptacle is used to capture, condense and store exhaust steam and any condensed steam which exits the sterilization unit. However, a single receptacle, which is limited in size since it must be emptied comfortably by a user, may be insufficient to receive and properly dissipate the exhaust steam and heat from the sterilization unit. The heat and steam from the sterilization equipment may build up in the area immediately surrounding the unit and the condensate receptacle, causing damage to the environment's shelving, and cabinetry or causing harm or discomfort to nearby users.

### SUMMARY OF THE INVENTION

Embodiments of the present invention provide an assembly and method for condensing exhaust heat and steam, including superheated steam, from a sterilization device.

In accordance with an embodiment of the present invention, there is provided an exhaust assembly for a sterilization apparatus. The exhaust assembly comprises a first exhaust vessel having a first inlet port, primary condensing means, a heat exchange media and a first outlet port, and a secondary exhaust vessel having a second inlet port connected to the first outlet port of the first exhaust vessel, secondary condensing means and a condensate exit.

In accordance with a further embodiment of the present invention there is provided an exhaust assembly for condensing steam. The exhaust assembly comprises a first exhaust vessel having a first inlet port, primary condensing means, a heat exchange media and a first outlet port, a second exhaust vessel having a second inlet port connected to the first outlet port, secondary condensing means, a condensate exit, exiting within the second exhaust vessel and a condensate outlet, and drainage means connected to the condensate outlet for draining condensate from the second exhaust vessel.

In accordance with a further embodiment of the present invention, there is provided a method of condensing exhaust steam from a sterilization apparatus using an exhaust assembly. The exhaust assembly comprises a first exhaust vessel having a first inlet port, primary condensing means and a first outlet port. There is a second exhaust vessel having a second inlet port connected to the first outlet port, secondary condensing means, a condensate exit, exiting within the second exhaust vessel, a condensate outlet and a condensate conduit, connected to said condensate outlet and extending within said second exhaust vessel. Drainage means is provided, comprising a drain tube and an air vent. The drainage means is connected to the condensate outlet for draining condensate from the second exhaust vessel. The method comprises the steps of: filling the first exhaust vessel with a heat exchange media; filling the second exhaust vessel with water to a level above the condensate intake end of the condensate conduit in the second exhaust vessel to prevent exhaust steam from exiting said second exhaust vessel via the intake end of said condensate conduit; passing exhaust steam from the sterilization apparatus to the first exhaust vessel; at least partially condensing exhaust steam in the first exhaust vessel, creating a mixture of exhaust steam and condensate; conveying the mixture of exhaust steam and condensate to the second exhaust vessel; condensing the mixture of exhaust steam and condensate, creating condensate, and draining the condensate from the second exhaust vessel. Air is vented from the drainage means preventing water from siphoning out of the second exhaust vessel to a level below the condensate intake end of the condensate conduit in the second exhaust vessel.

### BRIEF DESCRIPTION OF THE DRAWINGS

These and other advantages of the invention will become apparent upon reading the following detailed description and upon referring to the drawings in which:

FIG. 1 is a perspective view of an exhaust assembly in accordance with an embodiment of the invention connected to a sterilization apparatus.

FIG. 2 is a front view of an exhaust assembly, shown partially cutaway, in accordance with an embodiment of the invention.

FIG. 3 is a front view of an exhaust assembly, shown partially cutaway, in accordance with another embodiment of the invention.

FIG. 4 is a sectional view an exhaust assembly in accordance with FIG. 3, illustrating the draining of condensate.

While the invention will be described in conjunction with the illustrated embodiments, it will be understood that it is not intended to limit the invention to such embodiments. On the contrary, it is intended to cover all alternatives, modifications and equivalents as may be included within the spirit and scope of the invention as defined by the appended claims.

### DETAILED DESCRIPTION OF THE PREFERRED EMBODIMENTS

In the following description, similar features in the drawings have been given identical reference numerals where appropriate.

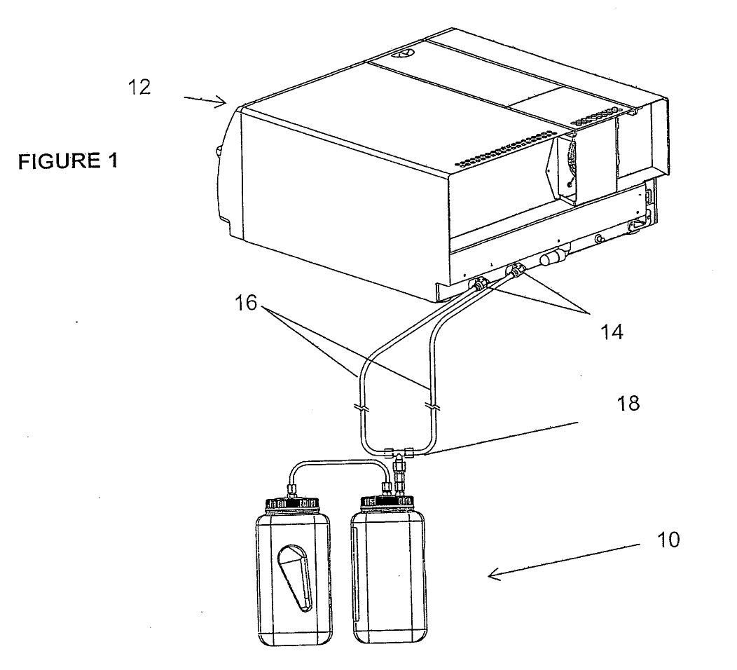

FIG. 1 illustrates an exhaust assembly 10 in accordance with an embodiment of the present invention connected to a sterilization apparatus 12, such as a cassette autoclave. Such sterilization apparatus 12, an example of which is sold by the applicant under the trade-mark STATIM and is embodied in numerous patents worldwide, including U.S. Pat. No. 5,271,893 of Newman, typically is used to sterilize medical or dental equipment (not shown). During the sterilization process, the sterilization apparatus 12 typically draws water from a supply reservoir (not shown) within the sterilization apparatus 12 to create steam and/or superheated steam for use during the sterilization process and to dry the sterilized equipment.

The exhaust assembly 10 of the present invention is suitable for use with the applicant's current STATIM CASSETTE AUTOCLAVE™ sterilizers but also with a sterilizer that uses superheated steam in the drying of the medical and dental instruments after sterilization. Such a sterilizer is described in the applicant's PCT Application No. PCT/CA2007/001197, the contents of which are incorporated herein by reference. It has been found that the volume of exhaust steam from the superheated steam sterilizer far exceeds that from the conventional sterilizer, thus necessitating a better exhaust handling system.

Exhaust, which may include condensate, steam, superheated steam and air, is vented from the sterilization apparatus 12 through one or more exhaust ports 14 to prevent moisture from remaining on the sterilized equipment contained within the apparatus. One or more exhaust ports 14 may be connected to the exhaust assembly 10. As shown in FIG. 1, two exhaust ports 14 may be connected to a single exhaust assembly 10 using exhaust tubes 16 and an interconnect 18. The exhaust tubes 16 typically may be comprised of polytetrafluoroethylene (PTFE) tubing, or any other suitable material for carrying high temperature exhaust in a flexible tube. The interconnect 18 typically may be comprised of nylon or any other suitable material for connecting the exhaust tubes 16.

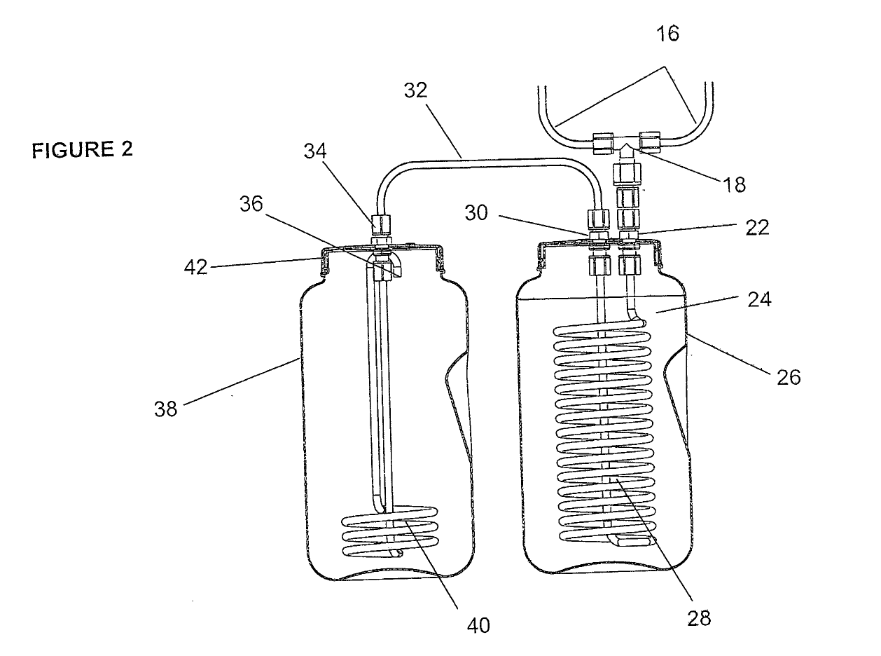

FIG. 2 illustrates the exhaust assembly 10 according to one embodiment of the present invention. The exhaust assembly 10 is comprised of a first exhaust vessel 26 and a second exhaust vessel 38. Exhaust tubes 16, carrying exhaust steam from the sterilization apparatus 12 are connected, via interconnect 18, to a first inlet port 22 of the first exhaust vessel 26.

The exhaust steam entering the first exhaust vessel 26 is condensed to a mixture of exhaust steam and condensate via a primary condensing means. As shown in FIG. 2, such condensing means may be a condenser coil 28 situated within a heat exchange media 24 within the first exhaust vessel 26. The primary condenser coil 28 typically is constructed of copper tubing and may be situated in heat exchange media 24 such as tap water. Typically, the first exhaust vessel 26 is initially filled with cool water in order to provide maximum heat exchange with the primary condenser coil 28. As required, the water or heat exchange media 24 is replaced with fresh water or other suitable media to prevent the growth of unwanted micro-organisms in stagnant water or other heat exchange media 24.

As the exhaust steam travels through the primary condenser coil 28, it is cooled and condensed to a mixture of condensed water and residual exhaust steam. The mixture of condensate and exhaust steam exits the first exhaust vessel 26 through a first outlet port 30 and is carried via an interconnect tube 32 to the second exhaust vessel 38. The interconnect tube 32 may be constructed of PTFE or any other suitable material for carrying high temperature exhaust and condensate.

As seen in the Figures, there is no mixing of the exhaust steam with the heat exchange media 24 in the first exhaust vessel 26.

The mixture of condensate and exhaust steam enters the second exhaust vessel 38 through a second inlet port 34. The residual exhaust steam in the mixture of condensate and exhaust steam is further condensed as it travels through a secondary condensing means in the second exhaust vessel 38. As seen in FIG. 2, the condensing means may be a condenser coil 40. Condenser coils 28, 40 may be constructed of copper tubing, or other suitable tubing intended for condensing coils may be used as appropriate. The condensate and any residual exhaust steam exit the secondary condenser coil 40 at the condensate exit 36.

As shown in FIG. 2, the condensate exit 36 may be located within the second exhaust vessel 38 wherein the condensate or waste water is stored in the second exhaust vessel 38 itself. As will be described in greater detail below, the condensate exit also may be connected to an exterior drainage means.

Typically, the size of the second exhaust vessel 38 corresponds to the size of the water reservoir in the sterilization apparatus 12 so that the second exhaust vessel 38 may hold at least as much condensate or waste water as the initial water reservoir supply. Thus, when the water reservoir of the sterilization apparatus is empty and needs to be refilled, the corresponding second exhaust vessel 38 is relatively full and can be emptied by a user at the same time. Although not shown in FIG. 2, the second exhaust vessel 38 may be detached from the exhaust assembly 10 by removing the cap 42 and condenser coil 40 to empty the condensate or waste water from the second exhaust vessel 38.

The first and second exhaust vessels 26, 38, may be any suitable container for storing the heat exchange media 24 and condensate or water, such as a polypropylene bottle. The vessels 26, 38 are of varying sizes but it has been found that a six-litre vessel functions well in that it provides a sufficient volume of heat exchange media in the first exhaust vessel 26 to effectively condense the majority of the exhaust steam for several hours of uninterrupted sterilizer use, yet it is an appropriate size to be handled by any user when the vessels 26, 38 need to be emptied and/or refilled. In operation, the vessels may be connected via an appropriate interconnect tube 32, such as PTFE tubing.

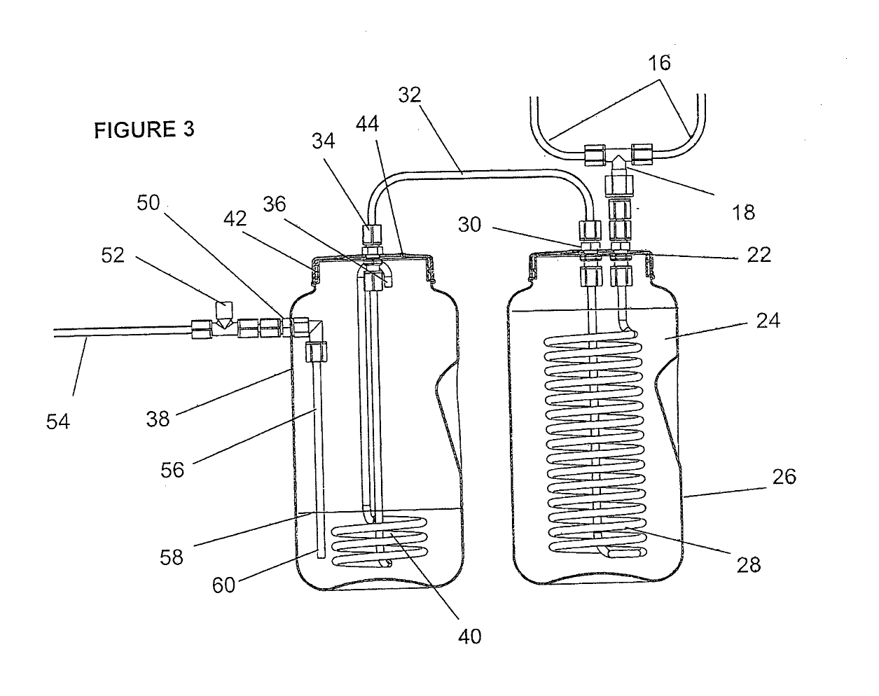

FIG. 3 illustrates a further embodiment of the present invention that further includes a drainage means in the second exhaust vessel 38 for disposing of the condensate or waste water directly to a sink or drainpipe (not shown). The second exhaust vessel 38 includes a condensate outlet 50 and a drain tube 54 for draining the waste condensate. As condensate collects in the second exhaust vessel 38, it may be drained via the condensate outlet 50 and drain tube 54.

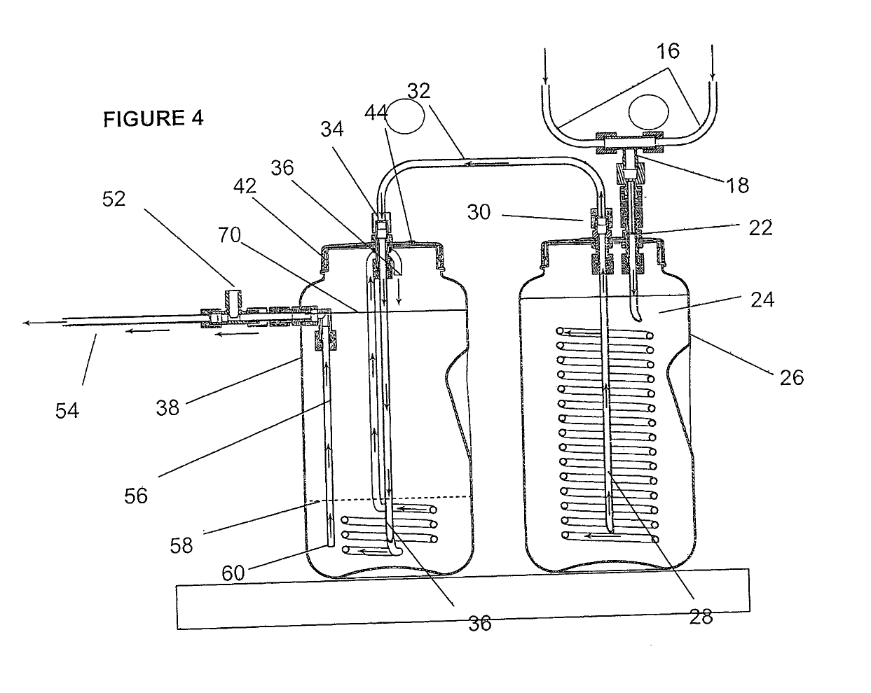

Since some building codes may prohibit the disposal of steam directly into a drainage system, it may be necessary to ensure that residual exhaust steam is prevented from entering the sink or drainpipe through the condensate outlet 50 and drain tube 54. In this case, in a further embodiment of the present invention, a condensate conduit 56 is provided which is connected to the condensate outlet 50 and which extends within the second exhaust vessel 38 below the condensate outlet 50. As shown in FIGS. 3 and 4, an air vent 52 in drain tube 54 allows for the venting of air to cause the condensate level inside the second exhaust vessel 38 to remain at the level of the condensate outlet 50.

In operation, the second exhaust vessel 38 is initially filled with a liquid condensate, such as tap water, to a minimum condensate level 58 above the condenser coils 36 and the condensate conduit intake 60. As shown in FIG. 4, as the level of condensate in the second exhaust vessel 38 reaches an upper condensate level 70 above the condensate outlet 50, the condensate will start to exit via the condensate conduit 56 and drain tube 54, starting a siphoning action. To prevent the condensate from being siphoned to the level of the condensate conduit intake 60, thereby potentially allowing exhaust steam to escape to the sink or drainpipe, air vent 52 ensures the condensate level does not fall below the level of the condensate outlet 50.

As the sterilization apparatus 12 progresses through the drying operation, a slight buildup in pressure may occur in the second exhaust vessel 38. An exhaust vent 44 may be provided in the cap 42 of the second exhaust vessel 38. Exhaust vent 44 may be used to control the air pressure in the second exhaust vessel 38 and to prevent an increase in air pressure from forcing the condensate level below the level of the condensate conduit intake 60.

The exhaust assembly of the present invention provides a significant improvement in condensing exhaust steam from a sterilization apparatus over a conventional single-vessel system for condensing and storing exhaust steam. A conventional system was tested using two single-vessel condensers inside a closed cabinet, each single-vessel condenser connected to one of two exhaust ports of a sterilization apparatus. After running back-to-back sterilization cycles for wrapped packages of equipment within the sterilizing apparatus, namely the cassette autoclave, for a duration of 120 minutes, the average relative humidity inside the cabinet housing the exhaust bottles was 65%, and the temperature reached a maximum of 52 degrees Celsius. Visual inspection revealed that the walls inside the cabinet contained a large build-up of moisture due to the high humidity, indicating that a significant amount of steam was not being condensed to water.

Using an exhaust assembly of the present invention, such as the embodiment illustrated in FIG. 1, after running back-to-back sterilization cycles for wrapped packages of equipment within the sterilizing apparatus, namely the cassette autoclave, for a duration of 120 minutes, the average relative humidity inside the cabinet housing the exhaust assembly was 35%, a 46% decrease from the conventional single-vessel system. The temperature inside the cabinet still reached over 50 degrees Celsius although it is anticipated that this arrangement will reduce the temperature in the cabinet over prolonged use in standard configurations. The improved condensation and disposal of exhaust steam via the exhaust assembly 10 of the present invention thus reduces the humidity and should reduce the heat in the area immediately surrounding the sterilization apparatus and exhaust assembly and thus reduces the risk of damage to furniture or other equipment in the immediate area and improves the user's comfort level should the vessels be kept in an open area.

Thus, it is apparent that there has been provided in accordance with the invention an apparatus for condensing and disposing of exhaust steam, such as from a sterilization apparatus, that fully satisfies the objects, aims and advantages set forth above. While the invention has been described in conjunction with illustrated embodiments thereof, it is evident that many alternatives, modifications and variations will be apparent to those skilled in the art in light of the foregoing description. Accordingly, it is intended to embrace all such alternatives, modifications and variations as fall within the spirit and broad scope of the invention.

1. An exhaust assembly for a sterilization apparatus, comprising:
a first exhaust vessel having a first inlet port, primary condensing means, a heat exchange media and a first outlet port; and
a secondary exhaust vessel having a second inlet port connected to the first outlet port of said first exhaust vessel, secondary condensing means and a condensate exit.

2. The exhaust assembly of claim 1 wherein the primary condensing means comprises a condenser coil situated in the heat exchange media, connecting the said first inlet port to said first outlet port.

3. The exhaust assembly of claim 1 wherein the secondary condensing means comprises a condenser coil situated in said second exhaust vessel, connecting said second inlet port to the condensate exit.

4. The exhaust assembly of claim 1 wherein the heat exchange media comprises water.

5. The exhaust assembly of claim 1 wherein the condensate exit empties into said second vessel.

6. The exhaust assembly of claim 1 wherein the condensate exit drains to a sink.

7. The exhaust assembly of claim 1 wherein said first outlet port is connected to said second inlet port by means of a tubing.

8. The exhaust assembly of claim 1 wherein said sterilization apparatus comprises a cassette autoclave for sterilizing medical and dental instruments.

9. An exhaust assembly for condensing steam, comprising:
a first exhaust vessel having a first inlet port, primary condensing means, a heat exchange media and a first outlet port;
a second exhaust vessel having a second inlet port connected to said first outlet port, secondary condensing means, a condensate exit, exiting within said second exhaust vessel and a condensate outlet; and
drainage means connected to said condensate outlet for draining condensate from said second exhaust vessel.

10. The exhaust assembly of claim 9 wherein said drainage means comprises a drain tube and an air vent located within said drain tube.

11. The exhaust assembly of claim 10 further comprising:
a condensate conduit within said second exhaust vessel, having a drain end connecting to said drainage means proximate to said condensate outlet and a condensate intake end extending below said condensate outlet.

12. The exhaust assembly of claim 11 wherein the primary condensing means comprises a condenser coil situated in the heat exchange media, connecting the said first inlet port to said first outlet port.

13. The exhaust assembly of claim 11 wherein the secondary condensing means comprises a condenser coil situated in said second exhaust vessel, connecting said second inlet port to the condensate exit.

14. The exhaust assembly of claim 11 wherein the heat exchange media comprises water.

15. The exhaust assembly of claim 11 wherein said first outlet port is connected to said second inlet port by means of a tubing.

16. The exhaust assembly of claim 11 wherein said sterilization apparatus comprises a cassette autoclave for sterilizing medical and dental instruments.

17. A method of condensing exhaust steam from a sterilization apparatus using an exhaust assembly, the exhaust assembly having a first exhaust vessel having a first inlet port, primary condensing means and a first outlet port, a second exhaust vessel having a second inlet port connected to said first outlet port, secondary condensing means, a condensate exit, exiting within said second exhaust vessel, a condensate outlet, a condensate conduit, connected to said condensate outlet and extending within said second exhaust vessel, and drainage means, comprising a drain tube and an air vent, said drainage means connected to said condensate outlet for draining condensate from said second exhaust vessel, said method comprising the steps of:
filling said first exhaust vessel with a heat exchange media;
filling said second exhaust vessel with water to a level above a condensate intake end of said condensate conduit in said second exhaust vessel;
passing exhaust steam from said sterilization apparatus to the first exhaust vessel;
at least partially condensing exhaust steam in the first exhaust vessel, creating a mixture of exhaust steam and condensate;
conveying the mixture of exhaust steam and condensate to the second exhaust vessel;
condensing the mixture of exhaust steam and condensate, creating condensate; and
draining the condensate from the second exhaust vessel wherein air is vented from the drainage means preventing water from siphoning out of the second exhaust vessel to a level below the condensate intake end of the condensate conduit in the second exhaust vessel.
